(19) United States
(12) Patent Application Publication
(10) Pub. No.: US 2017/0119833 A1
(43) Pub. Date: May 4, 2017

(54) ACANTHOSPERMUM HISPIDUM COMPOSITION FOR THE TREATMENT OF CANCER AND METHODS FOR ITS MANUFACTURE

(71) Applicants: DEEPA NATARAJAN, Chennai (IN); RAMESH T.S. CHOUHAN, Secunderabad (IN); SOPHIE MARIE HOFBAUER, Warszawa (PL)

(72) Inventors: DEEPA NATARAJAN, Chennai (IN); RAMESH T.S. CHOUHAN, Secunderabad (IN); SOPHIE MARIE HOFBAUER, Warszawa (PL)

(21) Appl. No.: 15/317,041

(22) PCT Filed: Jun. 10, 2014
(86) PCT No.: PCT/IB2014/062114

§ 371 Date: Dec. 7, 2016

(51) Int. Cl. A61K 36/28; A01G 1/00; A01G 31/02; A01G 9/14; A01K 63/06; A01G 7/04; A01K 61/10; A01K 47/00; A01K 63/04; A61K 31/216; A01G 9/24

(52) U.S. Cl. CPC A61K 36/28; A61K 31/216; A01G 1/00; A01G 31/02; A01G 9/14; A01G 9/24; A01G 7/045; A01K 61/10; A01K 47/00; A01K 63/042; A01K 63/06; A01K 63/045

(57) ABSTRACT

This invention relates to the field of cancer management, and in particular to the field of phytomedicine and nutraceutical composition for management of cancer, control of viral, bacterial and fungal infections, as well as other associated illnesses. This invention also pertains to the method of controlled, ecologically self-sustainable continuous horticulture combined with aquaculture (aquaponics system), for the described herbal plants and to the method for manufacturing the end products, both methods as per applicable ISO 14698 series and ISO-14644 series standards, EU GMP annex 1 and pharmacological Good Manufacturing Practice. The present application focuses primarily on Acanthospermum hispidum DC and on Solidago Canadensis L., and on their efficacy as anticancer agents, whereby 100% aqueous extract of Acanthospermum hispidum DC acts as immunomodulator possibly through initial vacuolisation followed by shrinkage of the cells and ultimately necrosis of the cells, and 100% aqueous extract of Solidago Canadensis L. exhibits the presence of cinnamoyl quinic acid that appears to cause the DNA groove binding thus preventing the formation of clumps of proteins which can distort the B-form double helix.

### TECHNICAL FIELD

This invention relates to the field of cancer management, and in particular to the field of phytomedicine and nutraceutical composition for management of cancer as well as other associated illnesses. This invention contemplates the use of the compositions alone or in conjunction with one or more known anti-cancer therapeutics as part of a combination therapy. This invention also pertains to the method of controlled, ecologically self-sustainable continuous horticulture for the described herbal plants and to the method for manufacturing the end products.

### BACKGROUND ART

The following references, which may include patents, patent applications and various publications, may be discussed in the description of this invention and are cited separately. The citation and/or citation of such references is provided merely to clarify the description of the present invention and is not an admission that any such reference is “prior art” to the invention described herein. All references cited or discussed in this specification are incorporated herein by reference in their entireties and to the same extent as if each reference was individually incorporated by reference.

Medicinal properties of a single compound or of extracts or fractions derived from herbs have thousands of years long history in traditional medicines all around the world. There is a growing international research activity in pharmaceutical community dealing with the multitude of plants that were tested and identified for anticancer activity which involves pharmaceutical effects of cytotoxicity, anti-proliferation, induction of differentiation, anti-inflammation, cell-cycle arrest, induction of apoptosis and/or autophagy, anti-invasion and anti-metastasis. Studies on efficacy of commonly used anticancer drugs shows that only 5 of the 25 listed drugs elicit preferential anti-solid tumor activity [1]. Furthermore, these agents have little impact on survival rates. This problem has been addressed in a contribution from medical oncologists from five continents, all arriving at the same conclusion on the inadequacy of current chemotherapeutic agents for the treatment of advanced solid malignancies [2].

The potential role of various plants in cancer therapy were investigated either for their direct anticancer activity, chemo preventive activity, radio-sensitizing activity or for immune enhancing activity. Several plants were evidenced to be used by aboriginal tribes as anticancer agents [3].

The present application focuses primarily on Acanthospermum hispidum DC; on Solidago Canadensis L.; on its compound Cinnamoyl quinic acid; and on their efficacy as anticancer agents, as well as for other health applications.

Acanthospermum hispidum DC (Bristly Starburr)

Acanthospermum hispidum DC, further referred to as “starburr”, (Family: Asteraceae, N.O. Compositae), is a hispid herb found as weed, originally from Brazil, which has spread throughout the world from Honduras and Nicaragua, southward to Argentina, it became naturalized in Africa, the Hawaiian Islands, India, Australia and the West Indies. In USA it is known throughout the southern states, and north up to New Jersey, it is considered a noxious weed invading plantations of peanut, corn and soybean and treated with herbicides.

The plant has been documented as diuretic, febrifuge, sudorific and in the treatment of gonorrhea and jaundice in some parts of South America. The plant also possess anti-viral activity against alpha herpes virus [4] and anti-plasmodial activity against plasmodium falciparum chloroquine resistant W₂ strains [5].

In-vitro studies revealed the immunomodulatory capacity of the plant to enhance the proliferation of T-Lymphocytes after stimulation with ConA or allogeneic stimulator cells in the mixed leucocyte culture [5]. The plant has also been reported for the presence of terpenoid and phenolic constituents, with some of the former possessing in-vitro antineoplastic activity [6]. Other species of this genus presenting cytotoxic and anticancer activity has been also documented [7]. Perhaps the most relevant observations and descriptions relative to the present patent application were made in the US patent [8] referring to the aqueous extract of starburr and in the previous work [9] of the present patent applicant.

Solidago Canadensis L. (goldenrod, Aaron's root or wound wort)

Solidago canadensis L., further referred to as goldenrod, is a genus of about 120 herbaceous perennial species of flowering plants in the Asteraceae family found mostly in the meadows, pastures, and waste areas of North America, Mexico, South America, Europe and Asia. In Germany it is considered an invasive species that displaces native vegetation from its natural habitat. The names “early goldenrod,” “European goldenrod,” and “Canadian goldenrod” are used interchangeably.

Goldenrod was reported and identified for its lipase activity, gastro protective property and employed as a component in urological phyto-therapy. It was shown to exhibit lipase activity with DNA polymerase β. Goldenrod has been tapped for phyto-constitutional investigation and was found to contain essential oil in almost all parts of the plant. Bayogenin glycoside, flavonoids, diterpenoids, saponins, lupine triterpenoid, germacrene-d-synthase and phenolic glycosides, were isolated from the aerial parts of the plant [10].

In-vitro effects of flavonoids on glutathione-s-transferase and cyto protective effect of diterpene solidagenone from the aerial parts of the plant is well documented [11]. In another study, clerodane diterpenes [12], were isolated from the roots of goldenrod and showed to have pronounced anti-fungal and nematicidal property, and photo-toxicity to certain viruses [14].

The roots of goldenrod were also found to have β-cadiene, ar-curcumene, β-caryophyllene, myricyl alcohol, cyclocolorenone as phyto-constituents in them. The aerial parts are reported for their diuretic activity. Goldenrod was also found to have caffeoyl quinic acid derivatives, ferulic and p-coumaric acids, sesquiterpenes. [13]. It has flavonoids, namely quercetin, kaempferol, glucopyranoside, rutin and their derivatives displaying strong anti-oxidant activity, and labdane diterpene [11] with gastro-protective effects. Goldenrod has been included in the herbal monograph and PDR for herbal medicines [15], for its use as diuretic, weak spasmolytic and anti-phlogistic properties.

### Cinnamoyl Quinic Acid

The cinnamoyl functionality is present in a variety of secondary metabolites of phenylpropanoid biosynthetic origin. Natural hydroxyl cinnamates are extremely potent class of antitumor agents. A lot of naturally occurring and biologically active alcohols have been linked with cinnamoyl residues through the ester linkage to amend their anticancer efficacy. Quinic acid nutritionally supports the synthesis of tryptophan and nicotinamide in the gastrointestinal (GI) tract, and this in turn leads to DNA repair enhancement and NF-kB inhibition via increased nicotinamide and tryptophan production [16].

### DEFINITIONS

Unless defined otherwise, all technical and scientific terms used herein have the meaning commonly understood by a person skilled in the art.

The term “plant material,” as used herein, refers to the specified parts of the plant harvested at the specified time and no other, due to either the toxicity of the plant part or to the specifics of propagation.

The term “plant extract”, refers to a composition prepared from plant material and a solvent following standard procedures described herein or understood in the art. The term encompasses crude extracts, prepared by a simple extraction, as well as crude extracts that have been subjected to one or more separation and/or purification steps. The plant extract may be in liquid form, such as a solution, concentrate or distillate, or it may be in solid form, such as in granulate or powder form.

The term “inhibit,” means to slowdown, reduce, delay or prevent.

The term “cell migration,” as used herein, refers to the movement, typically abnormal, of a cell or cells from one location to another.

The terms “therapy,” and “treatment,” as used interchangeably in the broadest sense, and include the prevention (prophylaxis), moderation, reduction, and curing of a disease, disorder or condition at various stages, and refer to an intervention performed with the intention of improving a recipient's status. The said terms apply to patients already having the disease, disorder or condition as well as those prone to, or at risk of developing, the disease, disorder or condition and those in whom the disease, disorder or condition is to be prevented.

The term “nutraceutical,” as used herein, refers to a food or dietary supplement that protects or promotes health and/or provides a benefit to a subject which affects the long term health of the subject.

The term “phytoceutical,” as used herein, refers to a plant-comprising composition having therapeutic properties.

The term “adjuvant,” as used herein, refers to substance that enhances and/or potentiates the therapeutic effect of another substance (such as a chemotherapeutic drug). In contrast, the term “adjuvant therapy,” as used herein with respect to cancer therapies, refers to a therapy that follows a primary therapy and that is administered to subjects at risk of relapsing. “Primary therapy” refers to a first line of treatment upon the initial diagnosis of cancer in a subject.

The term “in combination with”, as used herein refers to administration of the composition of the invention in combination with one or more further therapeutic agents, is intended to include simultaneous (concurrent) administration and consecutive administration intended to encompass administration of the therapeutic agent(s) and the composition(s) of the invention to the subject via various routes.

The term “about”, as used herein refers to a +/−10% variation from the nominal value. It is to be understood that such a variation is always included in any given value provided herein, whether or not it is specifically referred to.

### DESCRIPTION OF EXEMPLARY EMBODIMENTS

The present invention contemplates the use of the compositions alone or in conjunction with one or more known anti-cancer therapeutics as part of a combination therapy.

In one embodiment, this invention describes the method of obtaining 100% aqueous extract of starburr.

In another embodiment, there is a described dosage of the 100% aqueous extract of starburr for management of continuous vacuolisation and necrosis of the tumour growth and thus prevention of metastasis of vascularized tumours.

In another embodiment, there is a described dosage of the 100% aqueous extract of starburr for continuous anticancer immunomodulation and prevention of the viral, bacterial and fungal infections as well as for analgesic and anti-inflammatory effects.

In another embodiment, the 100% aqueous extract of starburr can be taken twice per week alongside the pharmaceutical prescription medication appropriate for the type of cancer.

In another embodiment, the 100% aqueous extract of starburr can be taken alongside 100% aqueous extract of goldenrod for combined effect of both nutraceuticals.

In another embodiment, the method for protected organic horticultural cultivation of starburr is described.

In another embodiment, the method for harvest, preparation, extraction manufacturing and packaging of final starburr product is described.

In another embodiment, this invention describes the method of obtaining 100% aqueous extract of goldenrod.

In another embodiment, there is described an effective dosage of the 100% aqueous extract of goldenrod for continuous inhibition of tumour growth through DNA groove binding and thus prevention of the complex protein formation that can result in gross distortions of the B-form double helix.

In another embodiment, the method for protected organic horticultural cultivation of goldenrod is described.

In another embodiment, the method for harvest, preparation, extraction and final product manufacturing and packaging is described.

Other embodiments propose possible compositions of individual or both extracts described herein with known and yet to be discovered anticancer therapeutics.

### DETAILED DESCRIPTIONS OF EXPERIMENTS

### Starburr

Starburr is an annual weed plant with toxic fruits which limits its use because it is difficult to control its best harvesting time, that is before flowering, to be useful for medicine. The entire plant, except the fruits is used. In the U.S. Pat. No. 5,256,416 [8], the authors described a rather complex method of isolating the water soluble toxic low molecular components (<2000). This patent is the only other officially described and animal tested method for aqueous extract from starburr preceding the one presented herein.

### Goldenrod

Goldenrod is a perennial plant, which for the purpose of a whole year production should be cultivated as partly annual, partly perennial. Only the entire previously planted part of the plant is used while the new roots and aerial parts are left for the next harvest.

There appears to be no precedent patent specifically involving goldenrod, its effectiveness in management of cancer and its side effects. On careful analysis of the literature documented so far it is evident that the plant and its genus have many promising phyto-constituents with varied pharmacological efficacies, like analgesic, antimicrobial, antifungal and antioxidant capabilities, which may be the solution for secondary problems of concern with patients afflicted with cancer. Hence the plant goldenrod is selected with an aim to explore its anti-cancer potential along with multi-protective pharmacological efficacies.

### Objectives of the Experiments

The experiments for starburr and for goldenrod were prepared and executed at the same time, using the same steps, therefore, to avoid repetition, the details are listed together with reference to respective plants as and where applicable.

In one embodiment, the objective is to refine the purity of previously described [8] aqueous extract and to find the pharmacological effectiveness of 100% pure aqueous extract of starburr without any other additives.

In another embodiment the objective is to find the chemical and pharmacological potentials of the plant goldenrod with focus on effectiveness of 100% pure aqueous extract of goldenrod without any other additives.

In another embodiment the objective is to isolate the cinnamoyl quinic acid and show its influence in preventing DNA corruption which may be the key factor in cancer growth.

### Collection and Preparation of Plant Materials

Starburr was collected complete, including flowers, prior to setting fruit. Goldenrod was also collected complete, including flowers, except only the previously plated parts of the plant are collected. Plants were separately air dried and ground to a coarse powder.

In one embodiment, starburr was extracted in an aspirated bottle with 100% double distilled water.

In another embodiment, goldenrod was extracted in separate aspirated bottle with ethyl acetate for further extraction of cinnamoyl quinic acid.

In another embodiment, goldenrod was extracted in separate aspirated bottle with 100% doubly distilled water. The extraction process started with cold maceration for 3-7 days. After decantation and filtering through whatman filter paper no. 41, nearly 80% of the solvent in each embodiment was removed by distillation over boiling water bath and remaining solvent was removed under reduced pressure.

In each embodiment, the extract so obtained was further dried in vacuum desiccator and the % yield of the extract was calculated.

The substances of interest, that is 100% aqueous extract of starburr gave 12.8% of residue, the 100% aqueous extract of goldenrod gave 10.72% of residue and the ethyl acetate residue gave 0.8% residue. The residue so obtained from these extracts was used for further studies.

### Plan of Experiments

Extracts were subjected to phyto chemical investigations (Qualitative chemical analysis, TLC and HPTLC finger printing).

Starburr aqueous extract was subjected to in-vitro anticancer study using DAL model. Antitumor activity of starburr was assessed by dead cell count, histopathology, hematological parameters, MST and body weight variation.

Goldenrod extracts were subjected to in-vitro anticancer study using DAL model and human cell lines (HeLa—cervical carcinoma cells and MCF-7—human breast cancer cells), in-vivo evaluation of the anticancer activity and those with more pronounced activity were further screened with in-vivo evaluation and in-vitro cytotoxicity by MTT assay, DNA binding activity and cell apoptotic studies.

The study was also designed to investigate the anti-microbial, antifungal, antioxidant, anthelmintic and analgesic efficacies of the extracts of both starburr and goldenrod plants using standard procedure. A detailed anatomy of the stem and leaves was used for identification of both plants. Lastly, the powder analysis and inorganic mineral analysis was performed to facilitate the identity and properties of the plants in final powder form.

### Antitumor Activity In-Vivo

The extracts used were in the form of suspension with tween-20 (one part by volume) and distilled water (two parts by volume). The prepared extracts were also used for further studies. Animals used for studies were Swiss albino mice. Six experiments were carried for each plant extract.

### For Goldenrod:

In one embodiment Group I was the tumor control;

In another embodiment, Group II was treated with 100% aqueous extract, 200 mg/kg. p.o.;

In another embodiment Group III was treated with 100% aqueous extract, 400 mg/kg.p.o;

In another embodiment Group IV was treated with standard drug 5-Fluorocil, 12.5 mg/kg.p.o.

### For Starburr:

In one embodiment Group I was the tumor control;

In another embodiment Group II was treated with 5 mg/kg.p.o. 100% aqueous extract;

In another embodiment Group III was treated with 50 mg/kg.p.o. 100% aqueous extract;

In another embodiment Group IV was treated with 300 mg/kg.p.o. 100% aqueous extract;

In another embodiment, Group V was treated with 2000 mg/kg.p.o. 100% aqueous extract;

In another embodiment Group VI was treated with standard drug 5-Fluorocil, 12.5 mg/kg.p.o.

After development of tumor the drug treatment was given on the 9th day and the 18th day post tumor inoculation. The peritoneal fluid was collected once on the 11th day and 20th day from each mouse and the following parameters were examined in antitumor assessment:

Ascitic fluid for cell cytology, specifically dead cell count and histopathology. Smear of ascetic fluid stained with May-Grünwald reagent was observed for vacuolization and necrosis of tumor cells;

Hematological parameters, such as WBC, RBC, Hb, differential Leucocyte count;

Mean Survival time (MST);

Body weight (tumor volume). All parameters were examined with standard procedures.

Statistical analysis: The data for anti-tumor activity was analyzed by one-way ANOVA followed by Dunnett's ‘t’ test. P<0.05 was considered statistically significant.

Antibacterial and antifungal activity. Bacteria used: Staphylococcus aureus, cscherichia coli, klebsiella pneumoniae, pseudomonas aeruginosa, proteus vulgaris, salmonella typhi, salmonella paratyphi A, salmonella paratyphi B, coagulase negative staphylococcus aureus, candida albicans, enterobacter pylori, shigella, acinetobacter and serratia.

Antibacterial study medium: Mueller Hinton (M.H.) agar; reference standard: Ciprofloxacin (5 μg/disc); method: disk diffusion.

Fungi used: Aspergillus fumigatus, Penicillium chrysogenum, Mycobacterium gypseum, Tricophyton simi, Tricophyton mentagrophytes, Tricophyton rubrum, and Aspergillus niger.

Antifungal study medium: SDA (Sabouraud dextrose agar); reference standard: clotrimazole 125 μg/ml; method: tube dilution. The residue of plant extracts were dissolved and diluted with DMF to get 1000 μg/ml and used for study.

Analgesic activity. Method I: Acetic acid induced writhing reflex. Method II: Eddy's hot plate. Animals: Wistar albino mice (25-30 g) of either sex. Standard: Morphine 5 mg/kg orally. Materials: extracts were prepared in the form of 1% w/v goldenrod MS suspension p.o. and morphine 5 g/kg.p.o. and administered to animals.

Antioxidant activity. DPPH assay: no radical inhibition assay.

Anthelmintic activity. For goldenrod, six groups of approximately equal sized Indian earth worms were used: Group I—vehicle (1% gum acacia in normal saline); Group II—standard drug (Albendazole—conc. 25, 50, 75 & 100 mg/ml); Group III—100% aqueous extract respectively (conc. 25, 50, 75 & 100 mg/ml). For starburr Group IV—100% aqueous starburr extract was used with the same concentration. Observations were made for the time taken to paralyze/death of the individual worms. Paralysis occurs when the worms are immobile and their body colour fades.

### EXPERIMENTAL RESULTS

### Starburr and Goldenrod

For 100% aqueous extracts of starburr and goldenrod, the anti-tumor activity was assessed by acute oral toxicity study performed as per Organization for Economic Co-operation and Development-423 (OECD-423) guidelines. Dose fixation was done for in-vivo and in-vitro studies. The doses were 5 mg/kg.p.o, 50 mg/kg.p.o, 300 mg/kg.p.o, and 2000 mg/kg.p.o. No animals died in all doses and the results were the same for both extracts. The behavior of animals was observed and found as follows: The animals were active. Alertness, touch response, pain response, righting reflex, gripping reflex, pinnal reflex, corneal reflex were positive. Grooming, restlessness, aggressiveness, tremors, convulsions, writhing reflex were negative. Pupils, skin colour and respiration were normal.

TABLE 1
Effect of 100% aqueous extracts of goldenrod and
starburr on dead cell count of DAL in mice

| | | % of dead | | % protection against | |
|---|---|---|---|---|---|
| | | cells | | tumor | |
| SI. No | Group | 11^{th} day | 20^{th} day | 11^{th} day | 20^{th} day |
| 1 | Control (DAL) | 23 | 33 | — | — |
| 2 | 5-FU (Standard) | 35 | 51 | 52 | 54.54 |
| 3 | 100% aqueous | 38 | 54 | 65 | 64 |
| | extract of starburr | | | | |
| 4 | 100% aqueous | 38 | 59 | 65 | 79 |
| | extract of goldenrod | | | | |

The results in TABLE 1 showing dead cell count in mice, indicate that 100% aqueous extracts of goldenrod and starburr possess more anti-tumor activity when compared with the standard 5-Fluorouracil and that goldenrod extract possesses more anti-tumor activity than starburr extract. Specifically, 100% aqueous extract of starburr is 25% more effective on 11th day and 17% more effective on the 29^{th} day, however, 100% aqueous extract of goldenrod is just as effective as starburr extract in the 11th day, but almost 45% more effective than 5-FU and 23% more effective than starburr on the 20th day.

The results of histopathology of ascitic tumor in all groups treated with the 100% aqueous starburr extract, indicate that there was vacuolisation and necrosis of tumor cells induced by the starburr extract and the effects were comparable with that of standard 5-Fluorouracil, however, the degree of vacuolisation and necrosis was greater in comparison to 5-Fluorouracil. Additionally, the intensity of necrosis and vacuolisation was significant on 20th day post-tumor inoculation with starburr extract. The findings indicate that the starburr extract has anti-tumor activity possibly through initial vacuolisation followed by shrinkage of the cells and ultimately necrosis of the cells.

On the other hand, in all groups treated with the 100% aqueous goldenrod extract, the necrosis of the cells was substantially greater than in the groups treated with standard 5-Fluorouracil and also with starburr extract, especially on the 20th day, which would indicate another, more effective mechanism. To find out more characteristics of this mechanism, in DNA binding studies, ethyl acetate extract of goldenrod was used. The absorption spectral properties of ethyl acetate extract show intense absorption bands with maxima of 316 nm and 318.5 nm and the intrinsic binding constant Kb of 1.7×10⁵. These spectral characteristics suggest that some component in ethyl extract might bind to DNA by an intercalative mode due to strong stacking interaction between aromatic chromophore of the extract and the base pair of the DNA. The spectroscopic experiment showing cyclic voltammogram of ethyl acetate in the presence and absence of different concentration of DNA, indicates slight increase in the flow time of DNA with increasing concentration of the extract, which would indicate that the extract prefers to engage in DNA groove binding or surface binding with its overall size resulting in an increase in DNA viscosity rather than an intercalative DNA interaction.

That particular ethyl acetate fraction 46-70, (eluted with 15% ethyl acetate and 85% hexane) on concentration yielded 82 mgs of pure gummy yellow-coloured homogenous solid which was identified as cinnamoyl quinic acid with molecular weight=321.58 and chemical formula=C16H17O7.

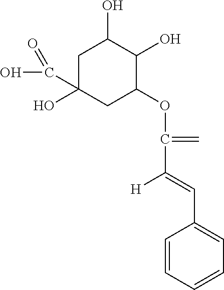 [SMILES: C=C(/C=C/c1ccccc1)OC1CC(O)(C(=O)O)CC(O)C1O]

This would indicate that it is the presence of cinnamoyl quinic acid in the goldenrod extracts that causes the DNA groove binding thus preventing the formation of clumps of proteins which can distort the B-form double helix.

TABLE 2
HPTLC of extracts of goldenrod

| Extract | No. of spots | Total height | Total area |
|---|---|---|---|
| 100% Ethyl acetate | 9 | 248.6 | 8477.1 |
| 100% aqueous extract | 12 | 1514.2 | 53217.8 |

The HPTLC comparison of goldenrod's 100% ethyl acetate extract and 100% aqueous extract shows that 100% aqueous extract is richer in other compounds than 100% ethyl acetate extract [TABLE 2], therefore it is the combination of all components of which cinnamoyl quinic acid is an important part preventing DNA corruption, but the action of other compounds are also responsible for degenerative changes in the form of tumour cell membrane blebbing, vacuolated cytoplasm and the tumour necrosis.

The acceptance criterion for determining the anti-tumor activity of a compound is the determination of circulating WBC as well as the life span prolongation. Applying this factor to the results of the hematological picture it is clear that the 100% aqueous extract of starburr as well as goldenrod significantly reduced WBC count. There is very little difference in effectiveness of both extracts. These findings establish the role of both extracts in exhibiting the anti-tumor activity. Both extracts appear to be more effective than the standard 5-Fluorouracil in controlling the WBC count by reducing the WBC count to nearly the value observed in normal healthy mice. Hematological changes are common in malignant disease and may be the presenting feature. The commonly encountered hematological changes in malignant disorders are anemia, sideroblastic anemia, leukocytosis, eosinophilia, monocytosis and thrombophlebitis.

TABLE 3
Effect of extract on changes in hematological parameters induced by DAL in mice

| | | WBC ± | RBC × | Hb g/dl ± | Lymphocytes ± | Neutrophils ± | |
|---|---|---|---|---|---|---|---|
| Description | Day | SEM | 10⁶ ± SEM | SEM | SEM | SEM | Monocytes |
| Normal mice | — | 5000-7000 | 8.7-12.5 | 10.2-16.2 | 70-75 | 23-28 | 0.1-3.5 |
| Tumor Control | 11 | 14,675 ± 1.86 | 3.25 ± 0.01 | 6.8 ± 0.97 | 56 ± 0.86 | 38 ± 1.13 | 2 |
| (DAL) | 20 | 16,633 ± 2.04 | 2.53 ± 0.02 | 6.5 ± 0.08 | 28 ± 0.81 | 58 ± 0.81 | 2 |
| 5-FU (Standard | 11 | 12,2375 ± 2.05 | 2.20 ± 0.08 | 5.8 ± 0.98 | 48 ± 0.41 | 56 ± 0.82 | 2 |
| 12.5 mg/kg. p.o) | 20 | 11,600 ± 2.03 | 2.61 ± 0.03 | 7.2 ± 0.04 | 54.98 ± 0.81 | 34.98 ± 0.03 | 2 |
| 100% Aqueous | 11 | 12,670 ± 2.12 | 2.49 ± 0.37 | 6.9 ± 0.04 | 43.25 ± 0.82 | 52.53 ± 1.64 | 2 |
| extract of | 20 | 8,250 ± 1.98 | 4.02 ± 0.04 | 11.98 ± 0.16 | 63.66 ± 0.12 | 28.16 ± 0.76 | 2 |
| starburr (300 | | | | | | | |
| mg/kg. p.o) | | | | | | | |
| 100% Aqueous | 11 | 12,470 ± 2.12 | 2.41 ± 0.37 | 6.9 ± 0.04 | 40 ± 0.82 | 52 ± 1.64 | 2 |
| extract of | 20 | 8,650 ± 1.98 | 4.02 ± 0.04 | 11.6 ± 0.16 | 61 ± 0.12 | 30 ± 0.76 | 2 |
| goldenrod (300 | | | | | | | |
| mg/kg. p.o) | | | | | | | |

A decrease in RBC count observed in the tumor control groups was more significant by the effect of both extracts particularly on 20th day post-tumor inoculation. This finding coincides with hemoglobin content. The mechanism responsible in malignant tumor for decrease in hemoglobin level followed by decrease in RBC count is complex and multifactorial. It could be due to shortened RBC survival, impaired bone marrow response to anemia, increased synthesis of ferritin, defective transfer of iron from reticulo-endothelial stores to RBC precursors and impaired transferrin production. Either or all of the mentioned mechanisms might be influenced by both extracts in affecting the RBC and hemoglobin picture and thus exhibiting the anti-tumor activity. Furthermore, the lymphocytes and neutrophil count were also controlled by both extracts. Both extracts seem to influence the hematological parameters in the anti-tumor activity to a greater extent than the standard 5-Fluorouracil.

TABLE 4
Effect of extract on mean survival time (MST) in mice

| | No. of Days | Mean Survival |
|---|---|---|
| Groups | Alive ± SEM | time (%) |
| Tumor control (DAL) | 22 ± 0.35 | 100 |
| 5-FU (Standard) | 26 ± 0.24 | 110 |
| 100% starburr | 57 ± 0.22 | 259 |
| aqueous extract | | |
| 100% aqueous | 61 ± 0.42 | 277 |
| goldenrod extract | | |

Prolongation of the mean survival time is a critical factor in determining the anti-tumor activity of a medication. The MST was extended to a greater extent by the starburr and goldenrod 100% aqueous extract treatment and the effect was much greater than that observed with standard 5-Fluorouracil. The mechanism for extended life span by the starburr extract treatment may be the influence of changes in the histopathology or hematology of the tumor cells and the mechanism for extended life span by goldenrod extract treatment may be also added by the DNA protection.

Tumor results in increase in body weight (TABLE 5), due to various factors listed above. The changes in body weight indicate that the starburr extract significantly reduced the tumor volume as compared to control or standard 5-Fluorouracil treated groups.

TABLE 5
Effect of extract on body weight (grams) of mice

| | | | | | | | | | | | | % increase in body |
|---|---|---|---|---|---|---|---|---|---|---|---|---|
| | | | | | | | | | | | | weight on 20th |
| | No. of days post tumor inoculation | | | | | | | | | | | day post-tumor |
| Groups | 9 | 10 | 11 | 12 | 13 | 14 | 15 | 18 | 20 | 21 | 24 | inoculation |
| Tumor Control | 24.5 | 24.5 | 25 | 25 | 26.75 | 30.5 | 31.5 | 34.33 | 34.33 | — | — | 40.83% |
| 5-FU Treated | 22.33 | 22.33 | 22.66 | 23 | 26.33 | 27 | 28.66 | 30 | 30 | 30 | 30 | 32.08% |
| These values are | 24 | 24 | 24.6 | 25.3 | 25.3 | 25.3 | 25.6 | 26.3 | 28.5 | 28.5 | 28.5 | 18.75% |
| common for 100% | | | | | | | | | | | | |
| aqueous extracts | | | | | | | | | | | | |
| of starburr and | | | | | | | | | | | | |
| goldenrod | | | | | | | | | | | | |

Each value represents the mean of six experiments

Comparison of the effect of the extract on dead cell count, histopathology, hematology, MST and body weight indicates that the findings observed in all these parameters agree with each other. An increase in dead cell count, or reduction in body weight representing anti-tumor activity is well reflected by the changes in histopathology or hematology of the tumor cells as seen with increased vacuolization and shrinkage of the cells followed by necrosis, in addition to decreased WBC count, increased RBC count, hemoglobin content and lymphocyte count and reduced neutrophil count, clearly prove that the starburr and goldenrod plants are a source of phyto-constituents with remarkably potent anti-tumor properties.

In-vitro cytotoxicity studies, MTT assay of 100% aqueous extract of goldenrod on human cell lines at 500 μg/ml concentration (Hoechst staining method), has shown to be more effective against HeLa cell line (42% apoptotic cells) then against MCF-7 cell line (30.33% apoptotic cells).

Compared to morphine (5 mg/Kg.p.o), the analgesic activity of 100% aqueous extract of goldenrod (400 mg/Kg.p.o.), was 61%.

The MIC antibacterial activity of 100% aqueous goldenrod extract in zone of inhibition compared favourably with standard ciprofloxacin, and in case of staphylococcus aureus, klebsiella pneumonia, acinetobacter baumannii and enterobacter aerogenes it was better than ciprofloxacin. Compared to standard clotrimazole (125 μg/ml), 100% aqueous goldenrod extract at 300 μg/ml exhibited remarkable 100% anti-fungal activity with all the tested organisms.

Analysis of anthelmintic activity of 100% aqueous goldenrod extract showed no signs of paralysis of pheretima posthuma, indicating that it protects the healthy intestinal flora and enhances the immune status of the patients afflicted with cancer.

Additional test (TABLE 6) of in-vitro cytotoxic activity performed with ethyl acetate extract and cinnamoyl quinic acid, shows that cinnamoyl quinic acid alone is responsible for the 82.35%, 76.36% and 84.21% cytotoxicity in HeLa, MCF-7 and DAL respectively.

TABLE 6
Goldenrod - comparison of cytotoxic activity of ethyl acetate
extract, cinnamoyl quinic acid and 100% aqueous extract

| | HeLa | MCF-7 | DAL |
|---|---|---|---|
| Test compound | (Ic 50 μg/ml) | (Ic 50 μg/ml) | (Ic 50 μg/ml) |
| Ethyl acetate | 170 | 275 | 190 |
| extract | | | |
| Cinnamoyl quinic | 140 | 210 | 160 |
| acid | | | |
| 100% aqueous | 205 | 240 | 290 |
| extract | | | |

Since cinnamoyl quinic acid is also contained in the 100% aqueous extract of the goldenrod, this comparison unequivocally points to a very prominent role of cinnamoyl quinic acid which in its pure form has that much effect. Furthermore, the anti-oxidant activity of cinnamoyl quinic acid is significant when compared to standard ascorbic acid. The cytotoxic and anti-oxidative properties of cinnamoyl quinic acid alone, marks it as a prominent therapeutically active component along with other constituents of goldenrod, exhibiting a potential role in the management and treatment of cancer and other associated illness like pain, infection, immune compromisation, etc.

### ADVANTAGEOUS EFFECTS

The 100% aqueous starburr extract exhibits better antitumor activity and it is simpler to prepare as well as it is chemically purer than other extracts described in U.S. Pat. No. 5,256,416 [8] and in applicant's previous work [9]. In addition to all the above mentioned healing properties, in the traditional herbal medicine, the starburr plant is claimed to also possess the immuno-modulatory, anti-microbial, anti-fungal, analgesic and anti-inflammatory activities. Since cancer normally affects immune response and facilitates infection by opportunistic pathogens, this necessitates use of anti-infective agents in cancer therapy. The indisputable fact is, that in large percentage of cancer affected population the currently available anti-cancer drugs cannot be used with inhibitors of other symptoms of this illness due to dangers of drug reactions. In contrast, starburr plant extract has a potent anti-cancer properties associated with anti-infective properties but without toxic effects, thus providing an unprecedented potential as cancer preventative as well as healing benefit to all cancer patients, regardless of a cancer type.

The 100% aqueous goldenrod extract applied in animal testing, unveiled anti-tumor effect against DAL cell line, increase in life span, decrease in average body weight and reversal of altered hematological parameters to near normal, which is a significant anti-tumor effect. The cytotoxic activity of 100% aqueous goldenrod extract exhibits remarkable effect against DAL and human cell lines (HeLa and MCF-7) by MTT assay and apoptotic studies.

Furthermore, the isolated example of cinnamoyl quinic acid which is inherent in the 100% aqueous goldenrod extract, showed strong DNA groove binding property through DNA binding assays using CT DNA fragments. Additionally, the goldenrod plant has parallel role in anthelmintic, analgesic and anti-infective activities, as these ailments are usually associated with cancer. In summary, goldenrod plant demonstrated potent anticancer activities similar to the standard drugs, but without the negative side effects and can be administered alone or concomitantly for greater effect.

The 100% aqueous extract of starburr and 100% aqueous extract of goldenrod are not only complimentary to the standard drugs, but also to each other. Historical evidence observed by the applicants in random volunteers experiencing different types of cancer and taking the nutraceuticals described here, shows that regardless of the type of cancer, the cancer cells progressively stop replicating after 45-60 days when the 100% aqueous extract of starburr or goldenrod is applied alongside the prescription chemotherapy. Furthermore, the negative health side effects are minimized and eventually eliminated.

The choice which extract to administer, depends on the stage of the cancer, the health state of the subject, the type of cancer and whether the chemotherapy or radiation therapy was used. The oncologists skilled in the administration of cancer controlling therapies and knowledgeable in the application of the nutraceuticals described here need to continuously evaluate the patients and adjust the intensity of the therapy accordingly.

It is an important consideration that 100% aqueous starburr extract's key property is immunomodulation possibly through initial vacuolisation followed by shrinkage of the cells and ultimately necrosis of the cells, while goldenrod extract's key property is DNA groove binding and prevention of distortions of the B-form double helix.

Both, starburr and goldenrod can be administered as prophylactic to people with exposure to cancer inducing environments such as carcinogenic chemicals and radiation. This is especially useful in situation of chemical or nuclear disaster. The prophylactic dose depends on severity of exposure and patient's weight and age.

In one embodiment, the prophylactic dose is 100 mg dose of starburr extract powder once a day as long as the environmental danger persists. Medical supervision is necessary and appropriate tests shall be made.

In another embodiment, the curative dose is 500 mg of goldenrod extract powder two times per week, depending on the results of cancer detection tests. The dose may be minimized as the number of cancer cells diminish.

In another embodiment, as continuous support after the remission of cancer, one 500 mg dose of starburr and one 500 mg dose of goldenrod once a week depending on the results of cancer detection tests. The dose may be minimized as the number of cancer cells diminish.

In another embodiment, the present and future cancer drugs may be supplemented with either starburr or goldenrod extracts or both, with separately administered doses according to the results of specific medical tests used in the art.

In another embodiment a therapeutic program may be created in combination with one or more anti-cancer therapeutics. In the context of the present invention, “anti-cancer therapeutics” include a wide variety of compounds, compositions and treatments that prevent or delay the growth and/or metastasis of cancer cells. Such anti-cancer therapeutics include, for example, chemotherapeutic drugs, radiation therapy, gene therapy, hormonal manipulation, immunotherapeutics, alternative therapy (including the use of other naturopathic preparations), and antisense oligonucleotide therapy comprising existing compositions and/or methods, and yet to be invented.

In another embodiment of the present invention, new compositions may be created in combination with one or more chemotherapeutic drugs. Suitable chemotherapeutics for use in combination with the therapeutic compositions of the invention can be selected from a wide range of cancer chemotherapeutic agents known in the art.

In one embodiment of the invention, the therapeutic compositions can be used in combination with one or more broad spectrum chemotherapeutics.

In another embodiments of the invention, the therapeutic combinations comprise specific chemotherapeutics.

The present invention further contemplates the use of a therapeutic compositions of the invention in combination with one or more immunotherapeutic agents. As is known in the art, immunotherapeutic agents can be non-specific, i.e. boost the immune system generally so that it becomes more effective in fighting the growth and/or spread of cancer cells, or they can specific, i.e. targeted to the cancer cells themselves. Immunotherapy regimens may combine the use of nonspecific and specific immunotherapeutic agents.

The present invention further contemplates the use of therapeutic compositions, for example as a nutraceutical formulation, in combination with one or more naturopathic preparations as part of a naturopathic therapy. For the purposes of the present invention, the term “naturopathic therapy” is intended to encompass various naturopathic, herbal, nutritional, botanical, homeopathic, alternative, and complementary therapies available for the treatment of cancer.

In accordance with the present invention, therapeutic compositions which are capable of inhibiting one or more of neoplastic cell migration, endothelial cell migration, tumour growth, and tumour metastasis are useful in the treatment of cancer. The activity of the compositions can be initially determined in vitro/in vivo if desired. A number of standard tests to determine the ability of a test compound or composition to inhibit cell migration, invasion and/or proliferation are known in the art and can be employed to test the plant extracts and therapeutic compositions. The inhibitory ability of combinations of therapeutic compositions and one or more anti-cancer therapeutics can be tested by similar methods.

Present historical evidence shows undoubtedly positive results in slowing the progression of cancer in human volunteers within six weeks of treatment and stopping it within 4-6 months of treatment, when combined in the treatment but applied separately with other appropriate chemotherapeutic regimen. Similar effects may be obtained with other products. Oncologists together with other specialists skilled in the art can design the best treatment program in accordance to age, sex and weight of the subject as well as to the stage of illness.

### BEST MODE

The modes described herein pertain to nutraceutical part of the invention. The in vitro/in vivo tests are needed for any combination of drugs and nutraceuticals (applied separately), and their influence on various cancers. Such tests shall be followed by clinical tests if the combination/composition becomes partly natural and partly synthetical pharmacological substance.

### Horticultural Considerations and Methods for Continuous Cultivation

Goldenrod and starburr grown in the wild should be avoided for several reasons: a) pollution from pesticides herbicides, fertilizers, chemical pollution from the environment it grows in (i.e. roads); b) if it grows in the forest or on pastures, then the uncontrolled harvesting may lead to the rapid eradication of the species, or alternatively, uncontrolled spread as both are considered as noxious weeds; c) starbur especially, must be collected prior to fruiting due to the fact that fruits are poisonous and removal of the fruits after the collection is too unreliable and risky; d) whether the plant is an annual (starburr) or perennial (goldenrod), the replication and growth needs to be continuous throughout the year for the manufacturing efficiency.

Because starburr grows very quickly (flowering in about 35 days), and because it grows in humid and subhumid areas in a southern and northern hemisphere, it is very suitable for year round greenhouse cultivation regardless of location. Starburr is a prolific seed producer with an average yield of over 4500 kg/ha of seeds [16]. The successive seeding of the plants should be best suited to the continuous production requirements.

Also the goldenrod, although a perennial growing in cooler areas, is also suitable for year round greenhouse cultivation. Goldenrod propagates best through its root rhizomes and frequent examination of roots is necessary to determine the best timing of propagation for continuous growth and blooming.

An establishment of honeybee colonies is recommended as a source of organic honey and for pollination of both plants. Nutrients in the honey may have their own protective effects and further studies may be in order.

For optimal growing conditions regardless of the climate, both plants should be grown in greenhouses for successive production, water, heating/cooling and shade, prevention of contamination as well as space management.

The best method of growth is hydroculture such as horticultural aquaponic method comprising hydroponics and intensive freshwater aquaculture which are mutually complementary. Because of the requirement of sufficient fresh water, a water purification system must be integrated in the fish farm which is much more efficient when it comprises water treatment and hydroponic horticulture, specifically, the continuous-flow solution method, where the nutrient solution constantly flows past the roots. It is much easier to automate than the static solution culture and has potential to serve thousands of plants. A popular variation is the nutrient film technique or NFT, whereby a very shallow stream of water containing the nutrients required for plant growth is recirculated past the bare roots of plants in a watertight thick root mat, which develops in the bottom of the channel and has an upper surface that, although moist, is in the air. Subsequent to this, an abundant supply of oxygen is provided to the roots of the plants while at the same the water from the fish pond is cleaned of the fish waste, which is sufficient to provide all the nutrients to the plants.

The fish pond should be populated at the same time as the plants are seeded and planted. The plants do not need the nutrients immediately, but until the levels build up from fish wastes, very small amount of organic nutrients may be needed for the plants, after which the aquatic animals do the work of maintaining proper plant nutrition. The composite fish culture system which is a technology developed in India by the Indian Council of Agricultural Research. Fish suitable for such aquaponic culture are tilapia, carp, catfish, clarias which can live in waters with up to 10% of fish biomass. However, in cold climates, the water in the fish water tanks may need to be heated as per the fish's requirements.

The growth area and the size of production facility should be calculated on the basis of proposed profitable annual output of the finished products. Such profitability may be enhanced by the commercial fish and honey production. Those skilled in the art of ecological self-sustainable horticulture, will readily adopt the above descriptions to the local conditions.

### Plant Material Collection and Extraction

In anticipation of the tightening global manufacturing standards for nutraceuticals to match those of pharmaceutical industry, the manufacturing of the products described herein is guided by pharmaceutical good manufacturing practices (GMP), which include cleanrooms, filtered air systems, aseptic filling techniques and cleanroom equipment to ensure safe manufacturing, and that the ingredients and dosages on the labels of nutraceuticals are accurate, and that finished products do not include any dangerous contaminants. The production method presented here is based on the specially designed, mostly robotic environment to provide precision and avoidance of errors. The proposed steps are broadly based on the legally binding pharmaceutical industry standards and are subject to the local/regional approvals.

The pharmaceutical grade GMP is applied throughout the greenhouse with aquaponic culture and throughout manufacturing facilities according to applicable ISO 14698 series and 150-14644 series standards and EU GMP annex 1. The reason for the greenhouse being also covered by the mentioned standards, is delivery of clean plants which are not contaminated with dust and dirt of the surroundings, as well as insects and microbes causing human and plant diseases. The same applies to the fish pond.

In one embodiment the whole plant of starburr, including roots, is harvested at the flowering time but before the fruiting time.

In another embodiment, only the previously planted part of the root system and the aerial parts of goldenrod are harvested at the flowering time, leaving the new root shots and the new aerial parts for the next harvest.

The plants must be kept fresh. Upon placing the plant singularly on the mowing inspection belt, the plant is inspected firstly by people. For added safety and quality, the single plants are then inspected by a proprietary multi-sensor system for optical precise recognition and knowledge based autonomous decision making, with plants not meeting the quality and safety specifications contained in the knowledge database, removed.

Useful plants are thoroughly spray washed twice with UV sterilized or ozonized clean water (without any trace of chlorine or fluoride), with excess water removed in vibrating separators, after which they are dried with 25°−30° C. dehumidified forced air combined with industrial bioconversion system delivering +/−99% air purification for removal of microorganisms, viruses, bacteria, mold spores and mycotoxins and bio-aerosols including ethylene.

When dried, the plants are pulverized. The pulverized plant in batches of 1 kg, is cold macerated with 100% doubly distilled water at 4° C. in an aspirated bottle for 4-7 days.

The extract is concentrated and dried. The techniques of solvent removal are known to those skilled in the art and include, but are not limited to rotary evaporation, distillation (normal and reduced pressure), centrifugal vacuum evaporation (speed-vac) and lyophilization.

In one embodiment, the yield is 12.8% of 100% aqueous starburr extract powder from each 1 kg of pulverized material. The batches are tested for conformity with the quality demands and when 99%-100% defect free, mixed together for uniform quality product.

In another embodiment, the yield is 10.72% of 100% aqueous goldenrod extract powder from each 1 kg of pulverized material. The batches are tested for conformity with the quality demands and when 99%-100% defect free, mixed together for uniform quality product.

Further processing is related to forming and packaging capsules with the extracts presented here. The packaging should be in sterile vacuum containers and should be defined by the variety of daily, weekly and full treatment doses described herein. After packing, the products shall be stored in clean room cold storage at 4° C. and transported to retail outlets in sealed boxes in refrigerated containers. Retail storage should be also under refrigeration with expiration date of 12 months.

### HEALTHCARE AND INDUSTRIAL APPLICABILITY

Nutraceuticals are a fast growing industry, which, when regulated on par with pharmaceutical industry, will gain great popularity in the market, as patients are more aware of the availability and benefits of pure, natural medicines and medicine that is alternative to the chemically derived drugs. The internet and social media spread the information and more doctors are also turning to natural solutions, especially that the research confirms the effectiveness of some of the nutraceuticals as being much greater than that of pharmaceuticals. As is evident from this patent application, the properly executed horticultural growth and process is similar in complexity to that of pharmaceuticals. The crude extracts have very small useful quantity per amount of the initial fresh, raw ingredient, as the herbaceous plants may have 70-95% of water content.

Assuming the average water content of 82.5%, then for 1 kg of dry plant powder, we need 8 kg of fresh plant. The plants can grow in layers narrow enough to allow the sunlight coverage, therefore the greenhouse is best designed as multilevel.

The patient needs on average 1 g of starburr extract per week and 1 kg of powder gives about 12 g of extract (considering production losses), or 12 weeks of medication. 1 kg of goldenrod powder gives about 10 g of extract (considering production losses), which gives 10 weeks of medication. According to historical evidence, the minimum time of treatment is about 16-25 weeks. Considering maintenance dose, the average patient's requirements is about 30-36 g of medication in the first year of treatment, after which only the maintenance dose is given.

The sequence of the text in any of the claims does not imply that process steps must be performed in a temporal or logical order according to such sequence unless it is specifically defined by the language of the claim. The process steps may be interchanged in any order without departing from the scope of the invention as long as such an interchange does not contradict the claim language and is not logically nonsensical. Skilled artisans may implement the described functionality in varying ways for each particular application, but such implementation decisions should not be interpreted as causing a departure from the scope of the present invention. While exemplary embodiments have been presented in the foregoing detailed description, it should be appreciated that a vast number of variations exist. It should also be appreciated that the exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function, material and arrangement of elements without departing from the scope of the invention as set forth in the appended claims and the legal equivalents thereof.

### PATENTS

U.S. Pat. No. 5,256,416, Oct. 26, 1993. Klauss Eichmann, Manuel Modolell: Extracts of the Acanthospermum Hispidum Plant.
China Patent CN101007037. Aug. 1, 2007. Zheng Shuiqing, Zheng Rong, Qin
Luping. Total triterpenes of solidago canadensis extracts, preparation method and use thereof.

### REFERENCES

1. Denis R. A. Mans, et al.: Anti-Cancer Drug Discovery and Development in Brazil: Targeted Plant Collection as a Rational Strategy to Acquire Candidate Anti-Cancer Compounds.
2. Sikora K., Advani S. Koroltchouk V. et al.: Essential Drugs for Cancer Therapy: a World Health Organization Consultation. Ann. Oncol. 1999; 10:385-390.
3. J. C. Th. Uphof: Dictionary of Economic Plants 2^{nd} Ed. Verlag Von J. Cramer, F,R.G. pages: 7, 272, 281 and 507 (1968)
4. Sanon S, Azas N, et al.: Antiplasmodial activity of alkaloid extracts from Pavetta crassipes (K. Schum) and Acanthospermum hispidum (DC), two plants used in traditional medicine in Burkina Faso. Parasitol Res. 2003 July; 90(4):314-7. Epub 2003 Apr. 4.
5. A. Summerfield, A. Saalmuller: Int. Immunopharmacology. 20(1-3) 85-88 (1998)
6. A. G. R. Nair, A. A. Rao, B. Voirin, J. Favre-Bonvin: Fitoterapia, 56, 249-50 (Elsevier, 1985)
7. A. A. Saleh, G. A. Cordell, N. R. Farnsworth: J. Chem. Soc. Perkin Trans-I 5, 1090-1097 (1980).
8. Klauss Eichmann, Manuel Modolell: U.S. Pat. No. 5,256,416, Oct. 26, 1993. Extracts of the Acanthospermum Hispidum Plant.
9. N. Deepa, N. N. Rajedran: Antitumor Activity of 50% Aqueous Ethanol Extracts of Avcanthospermum Hispidum DC in Mice. Natural Products: An Indian Journal August 2008.
10. Reznicek G. et al.: Four major saponins from Solidago canadensis. Pub. Med. Phytochemistry. 1991; 30(5):1629-33.
11. Marie-Eve Bradette-Hebert et al.: A New Labdane Diterpene From the Flowers of Solidago Canadensis Chem. Pharm. Bull. 56(1)82-84 (2008).
12. Apáti P, et al.: Herbal Remedies of Solidago—Correlation of Phytochemical Characteristics and Antioxidative Properties. J Pharm. Anal. 2003 Aug. 8; 32(4-5):1045-53. Elsevier Science.
13. Ravichandiran V. Deepa N.: In-Vitro Anticancer Activity of Solidago Canadensis L. (2012) Int. J. Res. Pharm. Sci. 3(1) 158-161.
14. N. Deepa, V. Ravichandiran: Antimicrobial Activity of Extractives of Solidago Canadensis L. (2010) Int. J. Res. Pharm. Sci. 1(4) 411-413.
15. Ram P. Rastogi: Journal of Scientific and Industrial Research, Volume 50 Issue 2 Central Drug Research Institute (India), Council of Scientific & Industrial Research (India). Publications & Information Directorate
16. Prithviraj De, M. Baltas, F. Bedos-Belval: Cinnamic Acid Derivatives as Anticancer Agents-A Review Current Medicinal Chemistry 2011, 18 1672-1703.
17. David W. Hall, et al.: Weeds in Florida, SP 37, Florida Cooperative Extension Service, Institute of Food and Agricultural Sciences, University of Florida. Publication date: May 1991. Rev. 2004, and February 2009.

We claim:

1. A composition of an extract where one component is starburr (acanthospermum hispidum).

2. A composition according to claim 1 where the second component is doubly distilled water.

3. A composition according to claims 1 and 2 that is a 100% crude aqueous extract, whose primary beneficial effect is immunomodulation, possibly through initial vacuolisation followed by shrinkage of the cells and ultimately necrosis of the cells with additional antiviral, antibacterial, antifungal, anti-inflammatory, analgesic and anthelmintic effects.

4. A composition of an extract where one component is goldenrod (solidago canadensis).

5. A composition according to claim 4 where the second component is doubly distilled water.

6. A composition according to claims 4 and 5 that is a 100% crude aqueous extract whose primary beneficial effect is DNA groove binding and prevention of distortions of the B-form double helix, with additional antiviral, antibacterial, antifungal, anti-inflammatory, analgesic and anthelmintic effects.

7. The composition according to claim 3 and 6 where the composition is formulated as nutraceutical, dietary supplement or naturopathic formulation for oral administration.

8. The composition according to claim 7 where the composition formulated for oral administration, may be in the form of a capsule containing pure 100% aqueous extract.

9. The composition according to claim 7 where the composition formulated for oral administration, may be in the form of a tablet or a capsule or as a syrup; any such composition formulated with commonly used organic/natural carriers such as organic/natural emulsifiers, thickeners, stabilizers, and texturant additives, for example acacia gum (Acacia senegal) or (Acacia seyal), tragacanth (genus Astragalus) and others, and organic sweeteners, for example honey, stevia (Stevia rebaudiana), and others.

10. A method for inhibiting tumour growth or metastasis by administering to the patient the composition according to claim 8, or 9 as a separate nutraceuticals complementing primary treatments or adjuvant therapies or to minimize their negative side effects.

11. A method where composition according to claim 3 or 6 is further purified as required for inclusion in a composition with one or more anticancer broad spectrum or targeted chemotherapeutic drugs or hormone therapeutic drugs or naturopathic, homeopathic or pharmaceutical substances or compositions.

12. A method according to claim 11 where the compositions are further tested in vitro/in vivo by the manufacturer for the optimal proportions of the compositions and dosage of the final products.

13. A method according to claim 12, where such compositions are complementing primary treatments or adjuvant therapies or to minimize their negative side effects.

14. A method according to claim 10, where the said compositions are administered as immunotherapeutics, alone or with one or more naturopathic or homeopathic preparations.

15. A method according to claims 10, and 14, where the said compositions are administered according to the stage of illness, age and weight of the patient.

16. A method of treating cancer using composition according to claim 15, where the patient is administered up to 500 mg of either composition, or of both compositions twice per week, subject to the results of applicable tests.

17. A method of prophylactic treatment with one or both compositions according to claim 15, in case of radioactive or chemical threat where the dosage can be 100 mg per day or greater, according to the severity of the exposure and the results of applicable tests.

18. A method of continuing post-recovery anticancer management with one or both compositions according to claim 15, where the dosage can be 100 mg per day or greater, according to results of the applicable tests.

19. A method where the component of extract according to claim 1 is obtained from the starburr plant harvested whole, in full bloom prior to fruiting.

20. A method where the component of extract according to claim 4 is obtained from the whole goldenrod plant harvested in full bloom with only the previously planted specimens removed and new roots and aerial parts left for the next harvest.

21. The starburr and goldenrod plants according to claims 19 and 20 are grown by aquaponic method.

22. The aquaponic method according to claim 21, is executed in temperature and light controlled greenhouses regardless of the climate of the location.

23. The aquaponic method according to claims 21 and 22, comprises edible fish aquaculture suitable for growing in temperate waters.

24. The aquaculture method according to claim 23 is a composite fish culture system allowing for greater biomass which provides all the necessary nutrients for the nourished plants.

25. The aquaculture method according to claim 22 includes fish ponds incorporated within the multilevel greenhouse architecture.

26. The aquaponic method of multilevel greenhouse architecture according to claim 25 incorporates plant growth area on bridges over the fish ponds and around the interior perimeter of the greenhouse.

27. The plants according to claims 1 and 4 are grown by continuous-flow solution method using the nutrient film technique (NFT).

28. The greenhouse and fish ponds comprising aquaponic method according to claim 26 are monitored with sensors for all the needs of plants and fish, with automatic adjustment to the ultimate conditions, including the need for oxygen, carbon dioxide and light.

29. The method of seeding the starburr plant according to claim 1, being an annual, is to plant seed every day according to the requirements of claim 27, after pretreatment for fast germination.

30. The method of harvesting the starburr plant seeded according to claim 29, is every 30-35 days, removing the whole plant when in full bloom, but before the fruit sets in.

31. The method for reseeding of starburr plants is according to claims 27 and 29, around the perimeter of the greenhouse away from the fish tanks, separating the seeded area with about one meter tall clear acrylic wall from the rest of the greenhouse to prevent accidental pollution of the fish pond or other plants with fruit or seed.

32. Honeybee colonies (apiculture) are required in greenhouses to pollinate the plants with hives located behind the separation as in claim 31.

33. Harvested starburr and goldenrod plants according to claims 19, 20 and 30 are placed singularly on the mowing inspection belt, with each plant inspected firstly by people.

34. For added safety and quality, the single plants are then inspected by a proprietary multi-sensor system for optical precise recognition and knowledge based autonomous decision making, with plants not meeting the quality and safety specifications contained in the knowledge database, removed.

35. Goldenrod plant according to claim 4, being a perennial, is planted as a rooted plant division according to requirements of claim 27.

36. The average time between planting and harvesting of goldenrod plant according to claim 35, is about 12-15 weeks.

37. About 4,500 goldenrod plant material, each having about 5 flowering stems and harvested according to claim 20, give about 1 kg 100% aqueous crude extract, which is sufficient for about 5 months of treatment for 50 patients.

38. About 5,500 starburr plant material and harvested according to claim 19, give about 1 kg 100% aqueous crude extract which is sufficient for about 1 year post-treatment immunotherapy or about 6 months prophylactic treatment for about 50 patients.

39. Either plant or both plants according to claims 37 and 38 may be used for treatment or post-treatment immunotherapy or prophylactic treatment subject to evaluation of the best treatment by oncologist.

40. The plants according to claims 33 and 34 are moved through the washing station where they are spray washed twice with clean water sterilized with UV or ozone, without chlorine or fluoride.

41. After washing according to claim 40, the excess water is removed in vibrating separators.

42. The drying process is with 25°-30° C. dehumidified ozone-free forced air combined with industrial bioconversion system removing +/−99% microorganisms, viruses, bacteria, mold spores and mycotoxins.

43. When dried according to claim 42, the plants are pulverized.

44. The pulverized plants in batches of 1 kg, are cold macerated with 100% doubly distilled water at 4° C. in an aspirated bottle for 4-7 days.

45. The extract from pulverized plants according to claim 44 is filtered, evaporated over water bath and dried in a desiccator as per standard practice.

46. The batches of finished extract according to claim 45 are tested for conformity with the quality demands and when 99%-100% defect free, mixed into larger batches.

47. Further processing is related to forming and packaging 100 mg capsules with the extracts presented here.

48. The packaging should be defined by daily and weekly intake and optionally include the instructions for use.

49. All tasks featured in claims 19 to 48 are performed according to applicable ISO 14698 series and 150-14644 series standards, EU GMP annex 1, and good manufacturing practices (GMP) for pharmaceutical industry.
